(19) United States
(12) Patent Application Publication
(10) Pub. No.: US 2023/0148258 A1
(43) Pub. Date: May 11, 2023

(54) SPERMIDINE-SUPPLEMENTED FOOD PRODUCTS

(71) Applicant: KALIN HEALTH LLC, New York, NY (US)

(72) Inventors: Stefano NATELLA, New York, NY (US); Robert LIEBERBERG, New York, NY (US); Allan KATZ, New York, NY (US)

(73) Assignee: KALIN HEALTH LLC, New York, NY (US)

(21) Appl. No.: 17/907,572

(22) PCT Filed: Apr. 6, 2021
(86) PCT No.: PCT/US21/26020

§ 371 Date: Sep. 28, 2022

Related U.S. Application Data
(60) Provisional application No. 63/006,677, filed on Apr. 7, 2020.

(51) Int. Cl. A61K 35/747; A61K 35/745; A61K 35/741; A61K 9/00; A61K 31/132; A23L 33/105; A23L 33/12

(52) U.S. Cl. CPC A61K 35/747; A61K 35/745; A61K 35/741; A61K 9/0053; A61K 31/132; A23L 33/105; A23L 33/12; A23Y 2300/21; A23Y 2300/49

(57) ABSTRACT

The nutritional food product compositions disclosed herein are provided to increase the levels of bioavailable spermidine. The compositions may include naturally-sourced spermidine in the form of a spermidine-containing fruit, vegetable, legume, and/or grain, one or more components that indirectly provides increased spermidine content, and a supplemental amount of spermidine. The compositions result in an improved profile panel of traditional biomarkers of general health and cellular health, higher levels of energy and alertness, reduced inflammation, and a stronger immune system.

This application claims the benefit of U.S. Provisional Application No. 63/006,677, filed Apr. 7, 2020, which is incorporated by reference herein in its entirety.

### FIELD OF THE INVENTION

This invention relates to a health-promoting food product for increasing bioavailable spermidine levels. The product may include naturally-sourced spermidine in the form of a spermidine-containing fruit, vegetable, legume, and/or grain, one or more components that indirectly provides increased spermidine content, and a supplemental amount of spermidine.

### BACKGROUND

Aging is a multifaceted process, caused by a myriad of interacting factors and with consequences at all levels of the organism. Some factors that contribute to the aging process include sustained exposure to cellular stress, chronic inflammation, increase in ROS (reactive oxygen species), dysregulation of lipid metabolism, and reduced autophagy (degradation and recycling of damaged cells). Effective interventions against aging will need to be able to impact as many as possible of the factors causing aging and their interactions.

Polyamines, especially spermidine, are involved in multiple facets of the aging process. Polyamines are polycationic molecules that interact readily with negatively charged molecules, including DNA, RNA, and lipids. Given that polyamines can interact with many molecules, it is not surprising that they affect aging via several mechanisms. Spermidine and the other polyamines (putrescine and spermine) have been shown to be involved in cell growth, maintenance and functioning. Spermidine has been reported to extend life span in model organisms including yeast, nematodes, flies and mice. Spermidine supplementation has also been reported to protect the heart and thus, prolong the lifespan of mice, while in humans it was correlated with lower blood pressure. It has also been found to slow aging in yeast, flies, worms, and human immune cells by inducing autophagy. Recent epidemiological evidence suggests that increased uptake of spermidine with food also reduces overall, cardiovascular and cancer-related mortality in humans.

It has been known for some time that polyamine levels decrease with age. Spermidine levels in humans between 60 and 80 years of age are lower than in humans below 50 years of age. However, healthy people who are 90 years of age and older have levels similar to people below 50 years of age. This suggests that maintaining spermidine levels in aging may contribute to health and longevity.

### SUMMARY

The compositions disclosed herein are designed to boost the levels of bioavailable spermidine. Increased levels of bioavailable spermidine can help support several critical cellular functions, improve health biomarkers, and promote anti-ageing health benefits to ultimately improve longevity and healthspan. The compositions disclosed herein are health food products with augmented spermidine content and at least one spermidine-boosting agent. The at least one spermidine-boosting agent is provided as a secondary means to increase bioavailable spermidine, in addition to the spermidine supplemented directly.

Some aspects of the disclosure are directed to a nutritional food product comprising a spermidine-containing fruit, vegetable, legume, and/or grain, an additional amount of at least one of spermidine and spermine, and at least one spermidine-boosting agent. In some embodiments, the at least one spermidine-boosting agent is at least one probiotic bacteria that synthesizes spermidine or supports the production of spermidine. Certain bacteria may support the production of spermidine by producing one or more compounds that lie upstream of spermidine in the spermidine biosynthetic pathway. In some aspects, the at least one probiotic bacteria is selected from the group consisting of Bifidobacterium animalis subspecies lactis LKM512, Lacidobacillus acidophilus, and Prevotella spp., or a combination thereof. In some embodiments, the at least one spermidine-boosting agent is a spermidine precursor. The spermidine precursor may be selected from arginine, ornithine, putrescine, or a combination thereof. The nutritional food product may include spermine, which is a biosynthetic product of spermidine. Providing supplemental spermine reduces the natural conversion of spermidine into spermine, and provides an additional means by which spermidine content can be increased. In some aspects, the composition further comprises at least one medium-chain triglyceride.

In some embodiments, the composition comprises spermidine in an amount ranging from 0.5 mg to 100 mg, preferably from 1 mg to 50 mg, more preferably from 5 mg to 10 mg. In some aspects, the composition comprises spermine in an amount ranging from 0.3 mg to 50 mg, preferably from 1 mg to 25 mg, more preferably from 3 mg to 5 mg. In some embodiments, each of the at least one probiotic bacteria is provided in an amount ranging from 1 Billion Units (BU) to 100 BU, preferably from 5 BU to 50 BU, more preferably from 10 BU to 30 BU. In some aspects, each of the at least one spermidine precursor is provided in an amount ranging from 50 mg to 5 g, preferably from 100 mg to 3 g, more preferably from 1 g to 2 g. In some aspects, the composition comprises a sugar content of less than 10 g, preferably less than 5 g. In some embodiments, the food product composition is provided in a serving size ranging from about 4 to 128 ounces or fluid ounces, or any value therein. In some embodiments, the spermidine-containing fruit is selected from mango, pear, apple, banana, strawberries, or a combination thereof. In some aspects, the spermidine-containing vegetable is selected from corn, cauliflower, spinach, broccoli, mushrooms, or a combination thereof. In some aspects, the spermidine-containing legume is selected from soy, peas, beans, lentils, or a combination thereof. In some aspects, the spermidine-containing grain is selected from wheat germ and wheat germ extract. In some aspects, the composition is a nutritional food product selected from the group consisting of milk, yogurt, gelatin, ice cream, sherbet, a shake, a smoothie, a powder for a beverage, a dairy-based beverage, and a non-dairy plant-based beverage. Non-dairy plant-based beverages include fruit juice, vegetable juice, fruit/vegetable juice, nut-based drinks, and energy drinks.

Some aspects of the disclosure are directed to a method for augmenting bioavailable spermidine level in a subject, comprising administering a food product composition comprising a spermidine-containing fruit, vegetable, legume, and/or grain, a supplemental amount of at least one of spermidine and spermine, and at least one spermidine-boosting agent. The increased bioavailable spermidine content has a beneficial effect on several core organs and tissues and improves several traditional bio-markers which are associated with a gradual decline of health conditions among older people. Some of the benefits that may be realized by increasing bioavailable spermidine content include some level of protection against the development of chronic diseases like cardiovascular diseases, prevention of mild cognitive deficit and Alzheimer's disease, promotion of cellular health and autophagy, lower incidence of diabetes, improved healthspan, and increased longevity.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. By “reduce or inhibit” is meant the ability to cause an overall decrease of 1%, 2%, 3%, 4%, 5%, 6%, 7%, 8%, 9%, 10%, 15%, 20%, 30%, 40%, 50%, 60%, 70%, 75%, 80%, 85%, 90%, 95%, or greater. Reduce or inhibit can refer to the symptoms and/or progression of a disorder or health markers associated with a disorder. By “improve” is meant to enhance or make better by an overall increase of 1%, 2%, 3%, 4%, 5%, 6%, 7%, 8%, 9%, 10%, 15%, 20%, 30%, 40%, 50%, 60%, 70%, 75%, 80%, 85%, 90%, 95%, or greater. Improve can refer to a reduction of symptoms or an increase of a positive indicator or outcome like blood pressure or heart rate. By “supplementing” is meant to increase the amount of.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, the methods and systems of the present invention that “comprises,” “has,” “includes” or “contains” one or more elements possesses those one or more elements, but is not limited to possessing only those one or more elements. Likewise, an element of a method or system of the present invention that “comprises,” “has,” “includes” or “contains” one or more features possesses those one or more features, but is not limited to possessing only those one or more features.

Any method or system of the present invention can consist of or consist essentially of—rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb. With respect to the phrase “consist essentially of,” a basic and novel property of the compositions and methods of the present invention is a nutritional food product containing a spermidine-containing fruit, vegetable, legume, and/or grain, an additional amount of at least one of spermidine and spermine, and at least one spermidine-boosting agent. Another novel property of the compositions and methods is the use of the composition to augment bioavailable spermidine concentration in a subject.

As used herein, in the specification, “a” or “an” may mean one or more, unless clearly indicated otherwise. As used herein, in the claim(s), when used in conjunction with the word “comprising,” the words “a” or “an” may mean one or more than one. As used herein “another” may mean at least a second or more. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a diagram outlining the polyamines biosynthetic pathway.

### DETAILED DESCRIPTION

Various features and advantageous details are explained more fully with reference to the non-limiting embodiments that are illustrated in the accompanying drawings and detailed in the following description. It should be understood, however, that the detailed description and the specific examples, while indicating embodiments of the invention, are given by way of illustration only, and not by way of limitation. Various substitutions, modifications, additions, and/or rearrangements will become apparent to those of ordinary skill in the art from this disclosure.

In the following description, numerous specific details are provided to provide a thorough understanding of the disclosed embodiments. One of ordinary skill in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The polyamines spermine, spermidine, and putrescine are involved in various biological processes, including cell proliferation and differentiation, and exhibit antioxidant properties. Spermidine and spermine are involved in maintaining chromatin structure and membrane stability in cells and regulating ion-channels and disposing of free radicals. They play a key transmission role in protein and nucleic acid synthesis for normal cell division and growth and contribute to tissue homeostasis of the gastrointestinal mucosa and epithelial cells. They inhibit oxidative stress, reduce atherosclerosis, and upregulate autophagy, leading to a much healthier and “younger” cellular make-up with reduced senescent cells. In addition, spermidine has been shown to protect against alpha-synuclein neurotoxicity, which is considered one of the main triggers of Parkinson's. The antioxidant and anti-inflammatory effects of polyamines are linked to their ability to induce autophagy, and play an important role in the prevention of chronic diseases such as cardiovascular diseases. In both animal and human models, spermidine intake is associated with increased longevity and some level of protection against the development of chronic diseases like cardiovascular disease, cancer, diabetes, and Alzheimer's. However, spermidine levels decline with age: spermidine levels are higher in healthy people who are 90 years of age and older, suggesting an association between spermidine levels and longevity.

Spermidine is a naturally-occurring polyamine that is present in all mammalian cells, suggesting that exogenous supply of this polyamine is unlikely to trigger any adverse effects. Spermidine is bio-available to mammals through the consumption of several foods and beverages which are part of a natural and healthy diet like fruits, vegetables, legumes, grains, and dairy-based and non-dairy plant-based products. Spermidine may represent one of the most attractive therapeutic approaches, among the agents that are known to induce autophagy. Published research has demonstrated that spermidine supplementation increases autophagy, which in turn boosts the immune response of older subjects. Alsaleh et al., eLife 2020, 9:e57950, p. 1-21. Specifically, Alsaleh et al. show that autophagy is activated in vaccine-induced antigen-specific CD8+ T cells in healthy young human volunteers but not in older subjects. Levels of the endogenous, autophagy-inducing metabolite spermidine fall in human T cells with age. Spermidine supplementation to aged donors allows T-cells to recover their autophagy level capability and function, similar to young donors' cells. These results link spermidine to positive effects on immune response and cellular and metabolic health. Furthermore, a number of studies demonstrate a correlation between high autophagy and good health outcomes, and low autophagy and bad health outcomes. For example, Fernandez et al., Nature 2018, v. 558, p. 136-140 and Sun et al., Circulation 2018, v. 138, p. 2247-2262. These studies provide a link between high autophagy (which is increased in response to spermidine supplementation) and protection against the onset of chronic diseases and increased healthspan.

Independent studies have determined that spermidine-rich wheat germ extract, a water-soluble extract of natural wheat germ that contains enriched levels of spermidine and spermine, is safe in a daily amount of 9.5 g of wheat germ extract, an amount that corresponds to a daily intake of 12 mg of spermidine and 6 mg of spermine. Food and Drug Administration GRAS Notice No. 889 Spermidine-Rich Wheat Germ Extract, the entirety of which is incorporated herein by reference. These levels of spermidine and spermine intake are far below the NOAEL (No Observed Adverse Effect Level) of 83 mg/kg body weight/day for spermidine (˜470 mg/day for a 70 kg person) and 1.52 mg/kg body weight/day for spermine (˜106 mg/day for a 70 kg person). Til et al., Food and Chemical Toxicology 1997, v. 35, p. 337-348.

A natural fruit, vegetable, legume, and/or grain-based food product with augmented spermidine content can be provided to healthy middle-age and older individuals in a way that emulates naturally-sourced spermidine. The food product includes a combination of natural fruit-, vegetable-, legume-, and/or grain-sourced spermidine and supplemented spermidine to provide a readily-absorbable source of spermidine. The food product may also include one or more spermidine-boosting agents selected from a spermidine precursor and one or more probiotic bacteria that support spermidine production. The food product may also include spermine. The food product is a natural, healthy, consumer-friendly, multi-source spermidine supplement that is especially beneficial for people 55 and older.

Ingested food is a major source of polyamines. The lumen in the upper part of the intestine absorbs most of these compounds to support cellular growth processes and autophagy throughout the body. Polyamines in the upper part of the intestine are transferred into the bloodstream via the colonic mucosa and reach all the different organs and tissues. Approximately 70-80% of spermidine and spermine are absorbed in the lumen and the remainder supports the development and maintenance of the digestive tract in two ways: nurturing the proliferation of the intestinal epithelial cells and enhancing the integrity of the intestinal barrier. The absorption of spermidine and spermine exhibits rapid kinetics; experiments on animal models have shown that close to 70% of the spermidine and spermine that reached the lumen were found in the portal vein just 10 minutes after.

The spermidine-supplementing compositions disclosed herein are provided to benefit consumer health. The compositions disclosed herein include up to four sources of spermidine; foods rich in spermidine, by-products of microbiota in the gut, cellular biosynthesis of spermidine from spermidine precursors via the polyamine biosynthesis pathway, and direct spermidine and spermine supplementation.

Spermidine and spermine are found in foods of both animal and plant origin in a wide range of concentrations. The food categories with the highest contents of spermidine and spermine are fruits, legumes, grains, and fermented milk. The soybean or soya bean (Glycine max) is a species of legume with high spermidine and spermine content. Various wheats of the genus Triticum are grains that are common sources of dietary nutrients and fiber. Wheat germ and wheat germ extract in particular have high values of spermidine and spermine. Mangos, pears, passion fruit, bananas, strawberries, as well as corn, peas, hazelnuts, almonds, pistachios, spinach, broccoli, cauliflower and mushrooms also contain significant amounts of spermidine and spermine. The compositions disclosed herein may include one or more spermidine and/or spermine-containing food or food product including but not limited to those identified here.

Intestinal microbiota is the collective name for the microbe population living in human intestines. The population includes tens of trillions of microorganisms, including at least 1,000 different species of known bacteria. The intestinal microbiota is also a major source of polyamines, including spermidine, and are the primary source of polyamines in the lower part of intestine, which get absorbed in the lumen or get excreted in the feces. Intake of certain beneficial bacteria, i.e., a probiotic, can lead to increased levels of intestinal spermidine and/or spermidine precursors. For example, intake of both Bifidobacterium animalis subsp. lactis LKM512 and arginine increases the synthesis of putrescine in the gut, a precursor of spermidine. The compositions disclosed herein may include one or more bacteria that are involved in synthesis of spermidine and/or spermine. The compositions disclosed herein may include one or more probiotic bacteria that are involved in the synthesis of spermidine and/or of a spermidine precursor. The compositions disclosed herein may include one or more compounds to support and/or stimulate growth of intestinal bacteria involved in the synthesis of spermidine and/or a spermidine precursor.

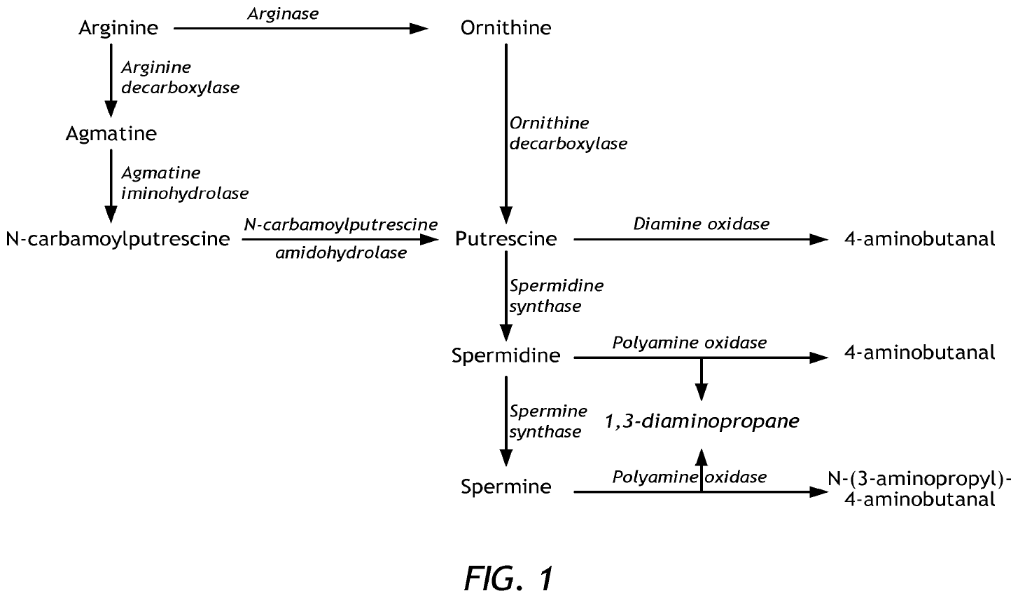

Spermidine precursors, spermidine, and spermine are synthesized in the body via the polyamine biosynthesis pathway (FIG. 1). Spermidine precursors putrescine, arginine, and ornithine are biosynthetically upstream of spermidine and spermine. Spermidine and spermine levels can be increased by supplementing these precursors. The compositions disclosed herein may include one or more spermine and spermidine precursors as a means to increase spermine and spermidine synthesis and levels.

Direct supplementation of spermidine and spermine provide a host of anti-aging and longevity benefits by promoting cellular health. Orally-ingested spermidine and spermine are readily absorbed by intestinal epithelial cells and distributed through systemic circulation without any noticeable degradation. The compositions disclosed herein may include spermidine and/or spermine in order to directly increase systemic spermidine and/or spermine levels.

The compositions disclosed herein may further include one or more medium-chain triglycerides. Medium-chain triglycerides help reduce inflammation and provide the brain with ketone bodies which boost the energy available to brain cells, and might help amplify the limited effect of spermine and spermidine supplementation on brain cells and reduce inflammation. Daily supplementation of medium-chain triglycerides prevents an increase in gut permeability and intestinal injury after endotoxin administration. Daily supplementation of medium-chain triglycerides can enhance intestinal secretion of Immunoglobulin A (IgA), an antibody that plays a crucial role in the immune function of mucous membranes. Intestinal secretory IgA is inversely correlated with bacterial overgrowth, bacterial translocation, and changes in intestinal permeability. Thus, intestinal secretory IgA plays an important role in protecting against infection in the intestinal immune system.

The term “medium-chain triglycerides” refers to triglycerides (i.e., tri-esters of glycerol and fatty acids) in which at least two of the three fatty acid moieties are derived from aliphatic (i.e., saturated open-chain) acids having between 6 and 12 carbon atoms, inclusive. The fatty acids that are used for making medium-chain triglycerides are defined as medium-chain fatty acids and include caproic (IUPAC, hexanoic), enanthic (IUPAC, heptanoic), caprylic (IUPAC, octanoic), pelargonic (IUPAC, nonanoic), capric (IUPAC, decanoic), undecylic acid (IUPAC, undecanoic), and lauric (IUPAC, dodecanoic) acids. Medium-chain triglycerides may be used in conjunction with the spermidine-supplementing compositions discussed above in order to provide added or synergistic benefits. Medium-chain triglycerides provide the brain with a doses of ketone bodies which boost the energy available to brain cells. This boost of energy may help amplify the limited effect of spermine and spermidine supplementation on the brain.

### EXAMPLES

Clinical Trial: A 3-months study of 20 subjects (10 control, 10 experimental) with placebo control, followed by an open-label phase of 3 months for all 20 subjects. The trial will assess the positive effect of the formula on anti-inflammatory, cognitive, metabolic and general health markers, and further explore selected biomarkers of cellular health, as well as safety linked to direct spermidine supplementation and increases in its bio-availability.

Study Methodology: Twenty individuals with a BMI in the 25-35 range, with no history of chronic diseases except diabetes, diabetic or non-diabetic, aged 40 to 75 years, will be randomly assigned to one of two groups. Group 1 will receive the formulation above with a daily doses of 7.5 mg of spermidine, 4 mg of spermine, 1.5 g of arginine, 20 BU of Bifidobacterium animalis subsp Lactis LKM512 and 7.5 g of medium-chain triglyceride oil served a 6 fl oz drinkable yogurt. Group 2 will receive a placebo of the formulations above containing either sugar, starch, or a combination of both for the first 3 months and the same as Group 1 in the following 3 months. Subjects will be studied 3 times, at baseline, at 3 months, and at 6 months.

End Points: Informed consent forms will be executed and participants will be monitored regularly for adverse events or side effects. Among the biomarkers to be tested at baseline, after 3 months, and 6 months are: serum beclin-1 (1-way ANOVA test), telomeres and telomerase, SPM-30, LC3 (cell health and autophagy); C-Reactive Protein, IL-6, TNF-α, adiponectin, apolipoprotein-E (inflammation and immune system); HbA1c (diabetes); DHEA, testosterone (hormones); blood pressure, heart rate, lipid panel (apoB/ApoA-I, LDL, TG/HDL), hemoglobin, hematocrits, bilirubin (cardiovascular health); AST, ALT, GGT (liver function); lactate and uric acid (mitochondrial dysfunction), antioxidant biomarkers like BAP assay, ROM assay, and thiol assay (oxidant status); Montreal Cognitive Assessment (MoCA), mini-mental state exam (MMSE), Mini-Cog, and ADAS-COG (cognitive health); and Groningen fitness test; senior fitness Test, AAHPERD, functional fitness test; walking speed, chair stand, grip strength, and standing balance (physical health).

The claims are not to be interpreted as including means-plus- or step-plus-function limitations, unless such a limitation is explicitly recited in a given claim using the phrase(s) “means for” or “step for,” respectively.

1. A nutritional food product, said food product comprising a spermidine-containing fruit, vegetable, legume, and/or grain, an additional amount of at least one of spermidine and spermine, and at least one spermidine-boosting agent.

2. The nutritional food product of claim 1, wherein the at least one spermidine-boosting agent is at least one probiotic bacteria that synthesizes spermidine or supports the production of spermidine

3. The nutritional food product of claim 2, wherein the at least one probiotic bacteria is selected from the group consisting of Bifidobacterium animalis subsp lactis LKM512, Lacidobacillus acidophilus, and Prevotella spp., or a combination thereof.

4. The nutritional food product of claim 1, wherein the at least one spermidine-boosting agent is a spermidine precursor selected from the group consisting of arginine, ornithine, putrescine, or a combination thereof.

5. The nutritional food product of claim 1, to further comprising at least one medium-chain triglyceride.

6. The nutritional food product of claim 1, wherein the spermidine is provided in an amount ranging from 0.5 mg to 100 mg.

7. (canceled)

8. (canceled)

9. The nutritional food product of claim 1, wherein the spermine is provided in an amount ranging from 0.3 mg to 50 mg.

10. (canceled)

11. (canceled)

12. The nutritional food product of claim 1, wherein each of the at least one probiotic bacteria is provided in an amount ranging from 1 BU to 100 BU.

13. (canceled)

14. (canceled)

15. The nutritional food product of claim 1, wherein the spermidine precursor is provided in an amount ranging from 50 mg to 5 g.

16. (canceled)

17. (canceled)

18. The nutritional food product of claim 1, wherein the food product is selected from the group consisting of a yogurt, gelatin, ice cream, sherbet, a shake, a smoothie, a juice, a powder for a beverage, a dairy-based beverage, and a non-dairy plant-based beverage.

19. A method for augmenting bioavailable spermidine concentration in a subject, the method comprising administering a food product comprising a spermidine-containing fruit, vegetable, legume, and/or grain, an additional amount of at least one of spermidine and spermine, and at least one spermidine-boosting agent.

20. The method of claim 19, wherein the at least one spermidine-boosting agent is at least one probiotic bacteria that synthesizes spermidine or supports the production of spermidine.

21. The method of claim 20, wherein the at least one probiotic bacteria is selected from the group consisting of Bifidobacterium animalis subsp lactis LKM512, Lacidobacillus acidophilus, and Prevotella spp., and combinations thereof.

22. The method of claim 19, wherein the at least one spermidine-boosting agent is a spermidine precursor selected from the group consisting of arginine, ornithine, putrescine, or a combination thereof.

23. The method of claim 19, further comprising at least one medium-chain triglyceride.

24. The method of claim 19, wherein the spermidine is provided in an amount ranging from 0.5 mg to 100 mg.

25. (canceled)

26. (canceled)

27. The method of claim 19, wherein the spermine is provided in an amount ranging from 0.3 mg to 50 mg.

28. (canceled)

29. (canceled)

30. The method of claim 19, wherein each of the at least one probiotic bacteria is provided in an amount ranging from 1 BU to 100 BU.

31. (canceled)

32. (canceled)

33. The method of claim 19, wherein the spermidine precursor is provided in amount ranging from 50 mg to 5 g.

34. (canceled)

35. (canceled)

36. The method of claim 19, wherein the food product is selected from the group consisting of yogurt, gelatin, ice cream, sherbet, a shake, a smoothie, a juice, a powder for a beverage, a beverage including dairy-based beverages and non-dairy plant-based beverages.
